(19) United States
(12) Patent Application Publication
(10) Pub. No.: US 2007/0134178 A1
(43) Pub. Date: Jun. 14, 2007

(54) Skin lightening complex

(76) Inventors: Iheatu Obioha, Santa Ana, CA (US); Tembi Sukuta, Rialto, CA (US)

Correspondence Address: LAWRENCE S. COHEN, SUITE 1220, 10960 WILSHIRE BLVD., LOS ANGELES, CA, 90024, US

(21) Appl. No.: 11/302,018

(22) Filed: Dec. 12, 2005

(51) Int. Cl. A61K 8/49; A61K 8/36

(52) U.S. Cl. 424/62

(57) ABSTRACT

A skin lightening complex having a skin lightening agent such as hydroquinone. The complex can be formulated in a concentrate form for later dilution as a product for topical application. The concentrate can be diluted such that the product is available as a pharmaceutical product with a high coincentration of active ingredients or as an over the counter product with a lower concentration. The concentrate is diluted with suitable carriers for the particular product. The formulation allows dilution of a high concentration of active skin lightening ingredient and has antioxidants and other ingredients for stable shelf life.

### FIELD

The invention relates to a complex for cosmetic and dermatological treatment, and to methods of making such a complex and methods of applying the complex.

### BACKGROUND

Topical skin-lightening products, such as lotions, creams, gels, sprays, etc are frequently used to treat skin disorders as age spots, melasma, chloasma, freckles, post-inflammatory hyperpigmentation or sun-induced pigmented blemishes. These skin-lightening products typically contain one or more depigmenting agents.

Depigmenting agents are chemicals that inhibit melanogenisis. Examples of depigmenting agents include but are not limited to hydroquinone, mequinol, kojic acid, arbutin, and licorice extract.

Hydroquinone (HQ), the most commonly used depigmenting agent worldwide is considered one of the best inhibitors of melanogenisis. HQ blocks melanogenisis by competitive inhibition of tyrosinase, the key enzyme in melanin synthesis. This agent may also inhibit DNA and RNA synthesis, degrade melanosomes, and destroy melanocytes.

HQ has high-level effectiveness and is a desirable depigmenting agent. HQ has a phenolic chemical structure and goes by a variety of other names, including 1,4-benzenediol; p-penzenediol; benzoquinol; 1,4-dihydroxybenzene; p-dihydroxybenzene; p-dioxybenzene; hydroquinol; hydroquinole; α-hydroquionone; p-hydroquinone; p-hydroxyphenol; quinol.

### DETAILED DESCRIPTION

The invention relates to a skin lightening complex for cosmetic and dermatological treatment, and to methods of making such a complex and methods of applying the complex. In one aspect the complex is prepared as a concentrate available for later dilution with one or more additives depending on the desired concentration of the active ingredient(s) and the type of application.

It is an object of the invention to provide a skin-lightening complex for topical application. In this aspect the complex will include a carrier used to allow convenient topical application.

It is a further object of the invention to provide a skin-lightening complex that has satisfactory shelf life for commercial purposes. In this aspect one or more ingredients are included that will act to prevent oxidation or other effects on the active ingredient(s) over time.

It is a further object of the invention to provide methods of formulating the skin-lightening complex into a wide variety of product types that include but are not limited to lotions, creams, gels, serum, sprays, wipes, and other such cosmetically or pharmaceutically acceptable topical forms. In this aspect a concentrate is made available for modification by addition of the appropriate ingredients for the desired end use.

Although the melanin-inhibiting effectiveness of HQ is desirable, water-based formulations containing high concentration HQ are difficult to prepare due to limitations in its solubility. HQ is soluble in aqueous media, sufficiently, up to 7.3 g/L at 25° C. As will be seen (see Example 2) the present invention allows the presence of about 20% HQ in the complex, which requires dissolving about 38.5 times more HQ in the solvent mixture. This is accomplished by using a solvent mixture having at least two solvents that have different polarity index.

HQ has been determined to be an unstable ingredient in formulations. HQ is easily oxidized in the presence of light, air, and water as well as by a variety of oxidants including oxygen in alkaline solutions. HQ undergoes redox chemistry, which produces oxidized products and as evidenced by darkening of the HQ containing products. Any brown discoloration of the product is an indication of deterioration in the strength of available HQ. An aqueous medium (water) contains reactive ions, which contribute to the powerful oxidizing properties. The hydroxyl ion OH⁻ is the most reactive ion. In an aqueous medium, the rate of HQ oxidation is pH dependent, occurring very rapidly at alkaline pH, leading to a brown solution. In acidic media the rate of HQ oxidation is very slow, suggesting that hydroxyl ions (OH⁻) play a key role in HQ oxidation.

HQ oxidation may be controlled by removing reactive ions and dissolved oxygen, which are present in water. The formation of oxidized products can be minimized or avoided by using an appropriate reducing agent, a chemical that can act as an electron donor.

The activity of skin lightening agents can be synergistically enhanced by an inclusion of additional active compounds such as antioxidants, alpha-hydroxy acids (or α-hydroxy acids) (AHA), beta-hydroxy acids (or β-hydroxy acids) (BHA), poly-hydroxy acids (PHA) etc.

This invention in one aspect relates to a concentrated liquid complex containing a dissolving depigmenting agent as an active ingredient, a reducing agent, an antioxidant, and an alpha-hydroxy acid, and the method by which it is produced.

In a related application, the skin lightening benefits of the composition is boosted by the inclusion of an antioxidant and an alpha-hydroxy acid.

This invention is directed to a skin lightening complex in the form of a liquid product with a pH of about 3.3 to about 4.5 and the method by which it is produced. The skin lightening complex contains a dissolved phenolic depigmenting agent such as hydroquinone (HQ).

The skin lightening complex of the current invention is preferably a topical skin lightening complex. The skin lightening complex may be manufactured as a concentrate with a high level of depigmenting agent such as Hydroquinone (HQ). It can then be appropriately diluted for any particular use. According to the present invention, a reducing agent such as sodium metabisulfite is used to control oxidation of the HQ.

The skin lightening complex of the current invention combines a highly effective phenolic depigmenting agent such as HQ with a peeling agent such as glycolic acid and an antioxidant such as ascorbic acid. These components enhance penetration and availability of HQ.

Another aspect of the invention relates to a method for dissolving a high concentration of a phenolic depigmenting agent such as HQ. HQ must be dissolved in order to be active. The method utilizes two miscible solvents each having a different polarity; the resulting solvent mixture has high solubility for HQ.

Another aspect of the invention is the formation of the skin lightening complex. In this aspect HQ is mixed into the solvent mixture, along with the reducing agent, preferably with optional heating, until completely dissolved. It is critical that the HQ be sufficiently dissolved in the solvent mixture. It requires about 60% to about 83% solvent mixture to obtain a dissolved concentration of HQ of about 20%. the preferred amount of the solvent mixture is about 70% (see Example 2). The antioxidant preferably hydroxy acid and glucolic acid are added at appropriate temperature until a clear mixture is obtained or to produce a uniform composition. This method of incorporating the HQ into the liquid base does not result in precipitation or oxidation of the HQ. After the HQ is dissolved in the solvent mixture along with the reducing agent and cooled, if heating is used, then the other components of the complex are slowly mixed into the resulting mixture.

In preparing the concentrate, hydroxy acid is then added. The hydroxy acid can be an alpa or beta or poly hydroxy acid.

The resulting concentration of ingredients in the concentrate are:

about 10% to about 30% by weight of the skin lightening agent as an active ingredient;

about 2.0% to about 3.6% by weight of reducing agent;

about 4.0% to about 6.0% by weight of an alpha or beta or poly hydroxy acid;

about 0.8% to about 1.2% by weight of antioxidant; and

about 60% to about 83% by weight of solvent mixture.

HQ has a melanin-inhibiting activity when it is completely dissolved in a solution having a pH of between about 3.0 to about 5.0. A high level of inhibiting activity has typically been reached between pH values in the range from about 3.0 to about 4.0. The pH of the skin lightening complex is adjusted to be between about 3.3 to about 4.5. Furthermore, the present composition does not lose melanin-inhibiting effectiveness at its formulated pH range.

Another aspect of the invention relates to methods of administering the skin lightening complex The skin lightening complex can be made into an emulsion (for example macroemulsions, microemulsions and nanoemulsions)/solution/gel suitable for topical application to skin and having physical and chemical stability for a prolonged period of time over a wide range of temperatures. For the purposes of providing a topical formulation with the skin lightening complex of the present invention, any of the known topical excipients can be used, including emollients, oils, emulsifying agents, preservatives, anti-oxidants, skin penetrants, etc.

The amount of the skin lightening complex of the present invention, which is to be incorporated into the topical formulations is an effective amount to lighten the skin.

By using the concentrate form of the skin lightening complex excipients and other carriers can be added for the particular use. In some cases the resulting product is a pharmaceutical with a higher concentration of skin lightening agent and other products are formulated as over the counter (OTC) and cosmetic products.

### EXAMPLE 1

Manufacturing Procedure for Skin Lightening

Hydroquinone is mixed into the solvent mixture, containing reducing agent, with optional heating, until it is completely dissolved. In a preferred procedure the reducing agent is mixed into the solvent mixture with heating and the HQ is added either with or without heating. If heating is maintained while dissolving the HQ it should not 60° C.

Other components of the composition including an antioxidant are slowly mixed into the solvent mixture and HQ under the appropriate temperature to produce uniform composition.

### EXAMPLE 2.

Product Evaluation.

Skin Lightening Complex.

SLC Batch #R147Gg prepared Dec. 31, 2003 by the formula

| Ingredients | % w/w |
|---|---|
| HQ | 20.00% |
| Ascorbic Acid | 1.00% |
| Glycolic Acid | 5.00% |
| Sodium metabisulfite | 2.75% |
| Propylene Glycol | 50.00% |
| DI Water (deionized water) | 21.25% |

Product was stored under RT (room temperature) and was tested on May 31, 2005.

Reference: ACCU Labs Ref 56133

Test Results:

Physical/Chemical Stability after 1.5 year RT

| Appearance: | homogenous transparent liquid | Confirm specification |
|---|---|---|
| Color: | yellowish | Confirm specification |
| Precipitation: | no | Confirm specification |
| pH: | 4.1 | Confirm specification |
| Assay HQ: | 19.892% | Confirm specification |

### EXAMPLE 3.

Application

The skin lightening complex prepared by the above procedure were followed in preparing topical lightening products of the following examples. The formulations of these examples illustrate the range of the invention. The examples all formed satisfactory products that are:
a) easy to prepare
b) significantly reduce manufacturing time
c) have sufficient stability.

### EXAMPLE 3.1

Skin Crème, Hydroquinone, 2%

| | |
|---|---|
| Skin lightening complex | 10.0 |
| Emulsifier | 3.5 |
| Co-emulsifier | 14.0 |
| Oil phase | 17.5 |
| Antioxidant | qs |
| Preservatives | qs |
| Fragrance | qs |
| Water | up to 100% |

Specification: Thick white cream @30C
   PH=3.9
   Viscosity @ 30C 21,000 cps
Assay testing: assay HQ%-1.97%—Reference: ACCU Labs Ref. # 56952 HPLC method

### EXAMPLE 3.2

Skin Lotion, Hydroquinone 2%

| | |
|---|---|
| Skin lightening complex | 10.0 |
| Emulsifier | 3.0 |
| Co-emulsifier | 9.5 |
| Rheology Modifier | 1.0 |
| Oil | 14.0 |
| Antioxidant | qs |
| Preservatives | qs |
| Fragrance | qs |
| DI water | up to 100% |

Specification: white lotion @30C
   PH=4.3
   Viscosity @ 30C 12,000 cps
Assay testing: assay HQ%-1.99%—Reference: ACCU Labs Ref. #56951 HPLC method

### EXAMPLE 3.3

Skin Gel, Hydroquinone 2%

| | |
|---|---|
| Skin lightening complex | 10.0 |
| SD Alcohol 40 | 15.0 |
| Polymer | 1.5 |
| Neutralizing Agent | 0.5 |
| Moisturizing Agent | 0.15 |
| Solubilizant | 0.5 |
| Antioxidant | qs |
| Fragrance | qs |
| DI Water | up to 100% |

Specification: transparent gel @30C
   PH=5.5
   Viscosity @ 30C
Assay testing: assay HQ%-1.99%—Reference: ACCU Labs Ref. #56953 HPLC method

### EXAMPLE 3.4

Skin Serum, Hydroquinone, 2%

| | |
|---|---|
| Skin Lightening complex | 10.0 |
| Polymer | 1.5 |
| Moisturizing Agent | 3.0 |
| Extract Botanical | 1.0 |
| Solubilizant | 0.05 |
| Preservative | qs |
| Antioxidant | qs |
| Fragrance | qs |
| pH adjuster | qs |
| DI Water | up to 100% |

Specification: white serum @30C
   PH=3.8-4.3
   Viscosity @ 30C 12,000 cps
Assay testing: assay HQ%-1.98%—Reference: ACCU Labs Ref. #56954 HPLC method

The foregoing Detailed Description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. This disclosure has been made with respect to the current state of the art, but also contemplates advancements and that adaptations in the future may take into consideration of those advancements, namely in accordance with the then current state of the art. It is intended that the scope of the invention be defined by the claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, component, or step is explicitly recited in the claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “step(s) for . . .”

What is claimed is:

1. A skin lightening complex comprising about 10% to about 30% by weight of lightening agent as an active ingredient; about 2.0% to about 3.6% by weight of reducing agent; about 4.0% to about 6.0% by weight of an alpha- or beta- or poly-hydroxy acid; about 0.8% to about 1.2% by weight of antioxidant; and about 60% to about 83% by weight of solvent mixture.

2. The skin lightening complex according to claim 1, wherein the lightening agent is selected from the group consisting of Hydroquinone, mequinol, kojic acid, arbutin, and licorice.

3. The skin lightening complex according to claim 2, wherein the lightening agent is hydroquinone.

4. The skin lightening complex according to claim 1, wherein the reducing agent is selected from the group of antioxidants that include water-soluble antioxidants such as sulfhydryl compounds and their derivatives.

5. The skin lightening complex according to claim 4, wherein the reducing agent is sodium metabisulfite.

6. The skin lightening complex according to claim 1 wherein the alpha, beta, poly-hydroxy acid is selected from the group consisting of alpha-hydroxy acids.

7. The skin lightening complex according to claim 6, wherein the alpha-hydroxy acid is glycolic Acid.

8. The skin lightening complex according to claim 1, wherein the antioxidant is selected from the group consisting of lipoic acid, lactoferrin, ascorbic acid and ascorbic acid derivatives.

9. The skin lightening complex according to claim 8, wherein the antioxidant is ascorbic acid.

10. The skin lightening complex according to claim 1, wherein the solvent mixture is selected from two solvents having different polarity index.

11. The skin lighting complex of claim 10 wherein the solvent mixture is selected from the group consisting of water, alcohol, propylene glycol, glycerol, and hexylen glycol.

12. The skin lightening complex according to claim 11, wherein the solvent mixture is a combination of deionized water and propylene glycol.

13. The skin lightening complex of claim 1 further comprising a sunscreen.

14. A skin lightening complex product comprising;
the concentrate of claim 1 further comprising a cosmetically or pharmaceutically acceptable carrier, preferably having a pH of between about 3 to about 5.

15. The skin lightening complex product of claim 14 wherein the carrier is any conventional carrier for topical administration and is employed in a concentration of about 98.5% to 75% by weight.

16. A method of preparing a skin lightening complex concentrate comprising;
preparing a solvent mixture comprising at least two solvents having a different polarity index;
adding to the solvent mixture a reducing agent;
dissolving in the solvent mixture containing the reducing agent, a skin lightening agent;
adding a hydroxy acid and an antioxidant;
wherein the resulting concentration of ingredients comprises the concentrations set out in claim 1.

17. The method of claim 16 wherein the step of adding the reducing agent is done with heating.

18. The method of claim 16 wherein the solvents of the solvent mixture are selected from the group consisting of;
ID water,
alcohol,
propolyne glycol,
glycerol, and
hexylen glycol.

19. The method of claim 16 wherein the solvents are deionized water and propylene glycol.

20. The method of claim 16 wherein the reducing agent is sodium metabisulfite

21. The method of claim 16 wherein the skin lightening agent is hydroquinone.

22. A method of preparing a skin lightening complex product comprising;
adding to the complex of claim 16 a carrier in a concentration selected for topical application having a pH of between about 3 to about 5.

23. The method of claim 22 wherein the carrier is in the concentration of about 98.5% to about 75% by weight.
